(19) United States
(12) Patent Application Publication
(10) Pub. No.: US 2008/0041396 A1
(43) Pub. Date: Feb. 21, 2008

(54) Mouth appliance for mitigating sleep related problems

(76) Inventor: David A. Lucker, Saint Joseph, MI (US)

Correspondence Address: JAMES RAY & ASSOCIATES, 2640 PITCAIRN ROAD, MONROEVILLE, PA, 15146, US

(21) Appl. No.: 11/893,968

(22) Filed: Aug. 17, 2007

Related U.S. Application Data
(60) Provisional application No. 60/838,210, filed on Aug. 17, 2006.

(51) Int. Cl. A61F 5/56

(52) U.S. Cl. 128/845; 128/848

(57) ABSTRACT

An oral nighttime appliance consists of a somewhat resilient U-shaped base substantially complementary with a person's dental arch. At least one integral lip protrudes from anterior edges of the U-shaped base, and a substantially rigid hourglass-shaped flap is connected into and partially spanning the gap between the posterior legs of the U-shaped base, and protrudes rearward and downward. A second flap, nearly semicircular and rigid, extends rearward and upward from the posterior of the base into which one end of the second flap is secured.

### CROSS REFERENCE TO RELATED APPLICATION

This patent application is related to and claims priority from U.S. Provisional Patent Application Ser. No. 60/838,210 filed Aug. 17, 2006.

### FIELD OF THE INVENTION

The present invention relates, in general, to sleep aids and, more particularly, this invention relates to mouth guards for minimizing habits and problems occurring during sleep time.

### BACKGROUND OF THE INVENTION

Many people suffer from routine sleeplessness due to breathing problems and sleep interruptions including grinding of teeth, snoring, or sleep apnea. A multitude of devices have been proposed over the years in an attempt to aid those suffering from the aforementioned afflictions. Generally, these have been devices for nocturnal wear either in the mouth or somewhere on the head.

Some of the prior art, such as Corniello in U.S. Pat. No. 3,132,647 take the approach of manipulating the tongue to create a more open airway through the mouth. The Corniello device engages just the upper teeth and incorporates a rather complicated tongue depressing pad construction. Others, such as Samelson in U.S. Pat. No. 4,304,227, aim to inhibit nearly all oral breathing, but Samelson still pulls the tongue forward so as to enlarge the air passage in the throat to facilitate nasal breathing. Those that inhibit breathing through the mouth are ignoring the common problem of clogged nasal passages that often accompany snoring. Nearly all of the previously proposed devices have U-shaped troughs designed to fit over many of the teeth in ones mouth. There are commercially available mandibular repositioning appliances for treatment of snoring and obstructive sleep apnea, such as that at www.somnomed.com, but none appear to be designed to hold the tongue down and away from the throat.

### SUMMARY OF THE INVENTION

The present invention provides an oral nighttime appliance consisting of a somewhat resilient U-shaped base substantially complementary with a person's dental arch. At least one integral lip protrudes from anterior edges of the U-shaped base. Connected into the posterior of the base is a substantially rigid hourglass-shaped flap partially spanning the gap between the posterior legs of the U-shaped base, and further protruding rearward and downward when the base is lying horizontally. A second flap, somewhat semicircular and rigid, extends rearward and upward from the posterior of the base to which it is also secured.

### OBJECTS OF THE INVENTION

It is, therefore, one of the primary objects of the present invention to provide a comfortable intra-oral device to prevent involuntary grinding of teeth while sleeping.

Another object of the present invention is to provide a nighttime mouth appliance that will prevent snoring.

Still another object of the present invention is to promote good unobstructed breathing during sleep and thereby reduce obstructive sleep apnea episodes.

Yet another object of the present invention is to minimize vibration of the soft upper palate while the user is sleeping.

In addition to the various objects and advantages of the present invention described with some degree of specificity above, it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a perspective view from one side of the mouth appliance of the present invention.

FIG. 2 is a frontal view of a person's head with the mouth appliance in place.

### DETAILED DESCRIPTION OF A PRESENTLY PREFERRED EMBODIMENT OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

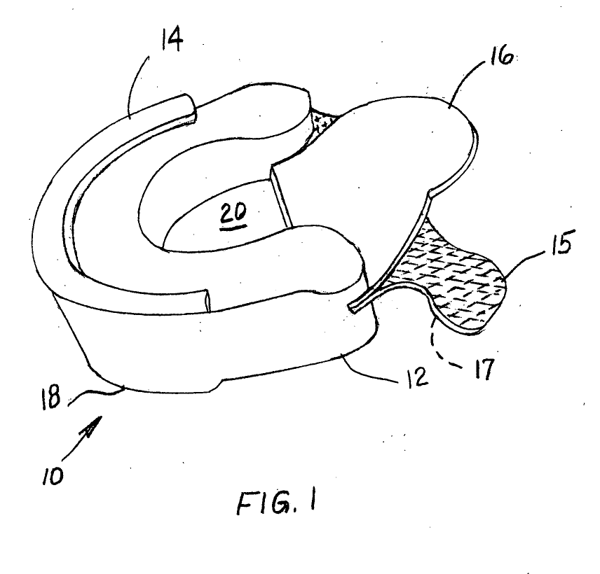

Referring initially to FIG. 1, the mouth appliance 10 is shown in perspective view. The U-shaped base 12 of the appliance is firm but resilient material that will fit comfortably over the lower teeth of the wearer. The upper anterior portion has a rim lip 14 and a similar lip 18 is along the bottom anterior edge. A somewhat rigid tongue depressor flap 15 is partially sandwiched about midway between the upper and lower surfaces of the base 12 at the posterior end. The tongue depressor 15 arches downward and has a textured surface 17 on the underside to help hold the tongue of the wearer away from the throat. This positioning of the tongue creates a more open air passage thus reducing the air velocity past the soft palate thereby minimizing the potential for snoring. Also helping to prevent snoring is an upward arching second flap 16 that contacts the posterior roof of the mouth. An open area 20 exists to allow the wearer's tongue to protrude through and touch the roof of the mouth. All parts are made of a material that can be shaped and molded when heated in hot water, but is essentially rigid at body temperature.

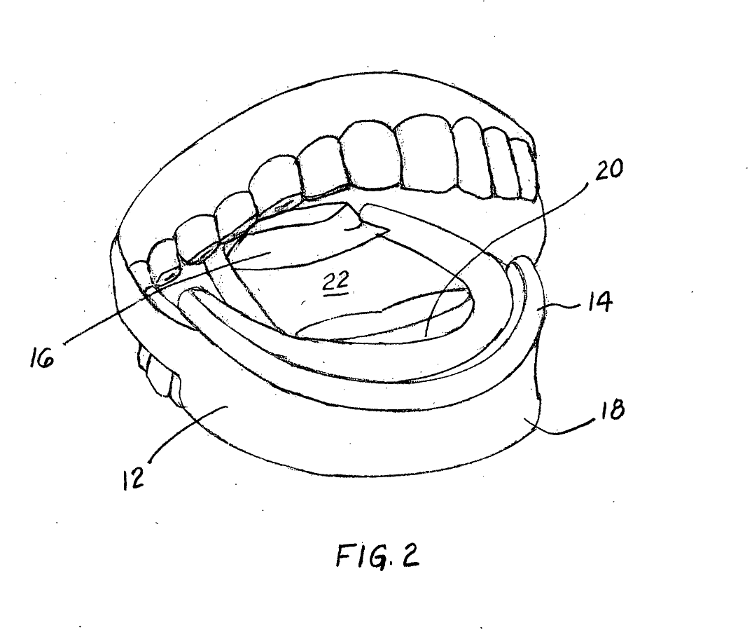

FIG. 2 is a frontal view of a wearer's head having the mouth open with the U-shaped base 12 engaged with the lower teeth. The bottom rim 18 extends down and outside the lower lip. When the mouth closes, the upper teeth rest on the U-shaped base just behind the upper rim. There is an opening 20 that allows the tip of the tongue 22 protrude through and touch the roof of the mouth when it closes. A somewhat rigid flap 16 arches upward and touches the roof of the mouth.

While a presently preferred embodiment of the present invention has been described in sufficient detail above to enable a person skilled in the relevant art to make and use the same, it should be obvious that various other adaptations and modifications can be envisioned by those persons skilled in such art without departing from either the spirit of the invention or the scope of the appended claims.

I claim:

1. An oral nighttime appliance comprising:
a) a resilient U-shaped base with a predetermined thickness and made of a preselected material, said U-shaped base substantially complementary with a person's dental arch;
b) at least one integral lip protruding from anterior edges of said U-shaped base;
c) a substantially rigid hourglass-shaped flap connected to said U-shaped base and partially spanning a portion of gap between two posterior legs of said U-shaped base, said hourglass-shaped flap protruding rearward and downward when said U-shaped base is substantially horizontal and;
d) a substantially rigid and substantially semicircular flap connected to said U-shaped base and partially spanning a portion of gap between two posterior legs of said U-shaped base, said semicircular flap protruding rearward and upward when said U-shaped base is substantially horizontal.

2. The oral nighttime appliance, according to claim one, wherein said preselected material is a thermoplastic elastomer.

3. The oral nighttime appliance, according to claim one, wherein said predetermined thickness is between about one centimeter and four centimeters.

4. The oral nighttime appliance, according to claim one, wherein said hourglass-shaped flap has a textured surface on the underside in an area in contact with a wearer's tongue.

5. The oral nighttime appliance, according to claim one, wherein said preselected material becomes moldable to a particular wearer's mouth and teeth after heating in hot water.
